SEQUENCE LISTING (4 sequences)

SEQ ID NO: 1 - protein, 539 aa, Drosophila sp.
MPSPTGVFVLMILTHLSLGLGQVRNEDQLLMVGQNGDLDSSNPAIHHQQHQQHQQHQQHQQHQSNHNLNNGNMNSTILNTLMGNNAGQVVNSSPGGGGSMINQLGSSTSSVPSVIGGGVGSVGNPWHSAVGLGVPGNGMGLPSSHGLGGNMGSHPHGHALAGLAKLGIIVPGGQGLPGNLGYGGTMLNGGGVGGAAGMGLRIGSNTNNMDMQQGLYNEHFISEHTVMAVFTSQGQVGGPCRYMPATRRQNHQCRKETGLPGTLSEARRLATTHCEEQFRYDRWNCSIETRGKRNIFKKLYKETAFVHALTAAAMTHSIARACAEGRMTKCSCGPKKHNREAQDFQWGGCNDNLKHGKRVTRSFLDLRGGDGDEVSEILRHDSEVGIEAVSSQMMDKCKCHGVSGSCSMKTCWKKMADFNATATLLRQKYNEAIAKAPNQRSMRQVSSSRMKKPKQRRKKPQQSQYTTLYYLETSPSYCAVTKDRQCLHPDNCGTLCCGRGYTTQVVKQVEKCRCRFNNGRCCQLICDYCQRVENKYFCK

SEQ ID NO: 2 - protein, 365 aa, Homo sapiens
MLDGSPLARWLAAAFGLTLLLAALRPSAAYFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 3 - protein, 357 aa, Homo sapiens
MRPPPALALAGLCLLALPAAAASYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 4 - protein, 537 aa, Homo sapiens
MAWRGAGPSVPGAPGGVGLSLGLLLQLLLLLGPARGFGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEVPLPHKTPIQPGEECHSVGTNSDQYIWVKRSLNCVLKCGYDAGLYSRSAKEFTDIWMAVWASLCFISTAFTVLTFLIDSSRFSYPERPIIFLSMCYNIYSIAYIVRLTVGRERISCDFEEAAEPVLIQEGLKNTGCAIIFLLMYFFGMASSIWWVILTLTWFLAAGLKWGHEAIEMHSSYFHIAAWAIPAVKTIVILIMRLVDADELTGLCYVGNQNLDALTGFVVAPLFTYLVIGTLFIAAGLVALFKIRSNLQKDGTKTDKLERLMVKIGVFSVLYTVPATCVIACYFYEISNWALFRYSADDSNMAVEMLKIFMSLLVGITSGMWIWSAKTLHTWQKCSNRLVNSGKVKREKRGNGWVKPGKGSETVV

(19) United States
(12) Patent Application Publication
(10) Pub. No.: US 2004/0072266 A1
(43) Pub. Date: Apr. 15, 2004

(54) Method of modifying cell motility via compounds and polypeptides that interact with a Wnt pathway

(76) Inventors: Elizabeth L. Wilder, North Potomac, MD (US); Ethan David Cohen, Philadelphia, PA (US)

Correspondence Address: Elizabeth L. Wilder, Ph.D., 14200 Pleasant Meadows Ct., North Potomac, MD, 20878, US

(21) Appl. No.: 10/251,769

(22) Filed: Sep. 23, 2002

(51) Int. Cl. G01N 33/574; C12N 15/85

(52) U.S. Cl. 435/7.23; 435/455

(57) ABSTRACT

The invention provides a novel method for analyzing cell motility by employing the Wnt signaling pathway. Cells and methods are described where Wnt polypeptides have a pronounced and measurable effect on cell motility. Agents that can be used to inhibit or induce cell motility can be screened for and identified.

### RELATED APPLICATIONS

This application claims priority to U.S. Provisional Application entitled Method of Facilitating Focal Adhesion Kinase-Mediated Cell Motility,” filed Sep. 21, 2001, and assigned serial No. ______, (Atty Docket No. 22253-69936) the entire contents of which are hereby incorporated by reference.

### GOVERNMENT RIGHTS

The U.S. Government may hold certain rights to this invention through its partial funding of the work related to this invention under a grant from the National Institutes of Health.

### INTRODUCTION AND FIELD OF THE INVENTION

The invention relates to, in one aspect, methods for identifying compounds that interact with wnt receptors or a wnt pathway to change cell motility characteristics in a cell. The invention also relates to the compounds identified through these methods and the methods of using these compounds to alter the cell motility characteristics of a cell. The invention also encompasses cells that can be used in these methods and cells with an altered cell motility characteristic compared to a control cell by virtue of the purposeful interaction with a wnt receptor or a wnt pathway. In another aspect, the invention relates to methods for altering the cell motility characetirstics of a cell through the manipulation of the wnt pathway in the cell. Additional aspects of the invention are described below.

Cell motility is regulated by extracellular cues and by intracellular factors that accumulate at sites of contact between cells and the extracellular matrix. One of these factors, Focal Adhesion Kinase (FAK), regulates the cycle of focal adhesion formation and disassembly that is required for cell movement to occur. Recently, Wnt signaling has also been implicated in the control of cell movement during vertebrate gastrulation, but the mechanism through which Wnt proteins influence motility is unclear. We demonstrate here that treatment with wnt polypeptides causes mammalian and vertebrate cells to dramatically increase the number of cell protrusions and/or alter the cell characteristics to increase cell motility. The interaction with the wnt pathway causes a change in the dynamic cellular structures involved with cell motility. Thus, cells that were previously adherant to or bound to other cells or within a tissue can become motile and other cells capable of movement display morphological changes in or changes in the protein level of certain motility-associated proteins the cell focal adhesions. A number of cytosketetal proteins or other proteins can be used to follow or measure the cell motility characteristic changes, including actin, FAK, PAK, src, paxillin, talin, vinculin, tensin, csk, crk, cas, pyk2, and integrins, for example. Any protein or polypeptide complex associated with or shown to be associated with focal adhesions, focal adhesion complexes, focal placques, focal complexes, and the like, or proteins or complexes shown to be involved in cellular attachment to an extracellular matrix, can be used to monitor or identify changes in cell motility characteristics. Simple and quantitative antibody-based assays can be used to detect or monitor changes in these protein levels or the morphological changes in protein expression.

We demonstrate that compounds that activate or functionally interact with the wnt pathway change the actin levels in mammalian cells and show that structural changes in the cell motility characteristics then become apparent. We have also generated mutations in Wnt4 and show that DWnt4 facilitates cell movement during ovarian morphogenesis. We demonstrate that DWnt4 is required for FAK accumulation in moving cells, and that it regulates FAK through a TCF-independent Wnt signaling pathway. We demonstrate that DFrizzled2 (DFz2) is the primary receptor for DWnt4 in this pathway rather than Frizzled (Fz), and the planar polarity proteins Prickled, Inturned, and Fuzzy are not required. The cell motility pathway is, therefore, distinct from both the canonical Wnt pathway and from the Fz-dependent planar polarity pathway.

Cell motility is critically important for processes as diverse as embryonic gastrulation and tumor metastasis. The regulation of cell motility requires the coordination of several signaling factors that impinge on the organization of the cytoskeleton (for reviews, see Critchley, 2000; Gumbiner, 1996; Lauffenburger and Horwitz, 1996; Parsons et al., 2000). Many of these factors are localized to focal complexes or focal adhesions, which serve to anchor the cytoskeleton to the extracellular surface and to provide traction as the plasma membrane extends forward. Cell movement begins with extension of filopodia or lamellipodia that are stabilized at the leading edge of the cell by focal complexes. As the cell surface continues to extend forward, these complexes become displaced from the leading edge and are referred to as focal adhesions. Focal adhesions are disassembled at the lagging edge of the cell, allowing the cell to move forward. The cyclical regulation of focal complex formation and disassembly is critical in the control of cell movement, and factors involved in this regulation are often localized to the complexes themselves.

Integrins are key components of these complexes, as they form the bridge between actin and the extracellular matrix. The cytoskeletal proteins vinculin, talin, and a-actinin are also present in focal complexes, as are several cytoplasmic protein tyrosine kinases, including Src and Focal Adhesion Kinase (FAK) (Critchley, 2000; Liu et al., 2000). The localization of these kinases to focal complexes leads to their activation and the subsequent phosphorylation of their targets. FAK phosphorylates focal adhesion components to induce focal adhesion disassembly. It can also function upstream of P13-kinase, Extracellular signal-regulated kinase (ERK) and Jun-N-terminal kinase (JNK) to regulate diverse cellular processes such as cell proliferation and viability (Parsons et al., 2000).

Recently, the Wnt family of secreted glycoproteins has been implicated or suggested in the regulation of cell movement. Wnt signaling is required during vertebrate gastrulation. However, a demonstration of cell motility charateristic changes directly through a Wnt protein and/or through the Wnt pathway had not been made and a mechanism through which Wnt proteins could facilitate cell movement had not been understood. Furthermore, no mutants in the model system of Drosophilia had indicated any direct action on cell movement as a result of a Wnt gene.

Wnt proteins form a large group of secreted ligands that regulate many cellular processes, including cell fate specification, cell proliferation, epithelial/mesenchymal transitions, cell adhesion, and cell motility (McEwen and Peifer, 2000; Patapoutian and Reichardt, 2000; Polakis, 2000). Many of these processes are mediated through a canonical pathway which ultimately results in the stabilization and nuclear translocation of b-catenin. In this pathway, Wnt proteins activate the cytoplasmic protein Disheveled (Dsh) through association with members of the Frizzled (Fz) and LDL-Receptor families (Cadigan and Nusse, 1997; Pinson et al., 2000; Wehrli et al., 2000; Tamai et al., 2000). Activation of Dsh results in inhibition of Glycogen Synthase Kinase 3b (GSK3b). In the absence of Wnt signaling, GSK-3b phosphorylates b-Catenin, targeting it to the proteasome for degradation. However, in the presence of canonical Wnt signaling, GSK3b is inhibited, so b-catenin accumulates and is translocated to the nucleus where it complexes with TCF family DNA binding proteins. This b-catenin/TCF complex regulates the transcription of target genes. Although this pathway mediates many Wnt-regulated processes, it does not appear to be involved in Wnt regulation of cell movement (Heisenberg et al., 2000; Tada and Smith, 2000; Wallingford et al., 2000).

The cellular and molecular mechanisms underlying Wnt regulation of cell movement are poorly defined. The Wnt signal transducer Dsh is required for the polarized stabilization of lamellipodia projected by dorsal mesodermal cells during convergence and extension (Wallingford et al., 2000). During this process, cells extend lamellipodia in all directions, but these projections are preferentially stabilized in what will be the direction of movement. In the presence of an interfering form of Dsh, cells randomly extend lamellipodia which fail to stabilize in the direction of movement. The ligand that mediates convergent extension during zebrafish and Xenopus gastrulation is Wntl 1 (Heisenberg et al., 2000; Tada and Smith, 2000). Wntl 1 mutations disrupt axis elongation in zebrafish and Xenopus embryos, and these effects can be rescued by Dsh. This indicates that Wnt proteins regulate lamellipodial behavior through Dsh.

The polarization of actin extensions from moving cells during gastrulation is reminiscent of the establishment of planar polarity in Drosophila. In the wing epithelium, this process involves the localization of actin bundles to the distal tips of hexagonally packed cells (Adler, 1992). In this tissue, the actin localization prefigures the formation of actin-based hairs rather than leading to cell movement. The similarity in actin organization between lamellipodia extension and hair formation suggests that the signal transduction pathways involved in these two processes are similar (McEwen and Peifer, 2000). This idea is supported by the observation that mutant forms of Dsh that abolish function in planar polarity, but not in canonical signaling, also interfere with Dsh activity during convergent extension (Tada and Smith, 2000; Wallingford et al., 2000).

Although the prior analyses suggest that the pathways used by Wnt proteins to facilitate cell movement and the planar polarity pathway are similar, it has been difficult to directly compare the two. No ligand has been identified in the planar polarity pathway, and Wnt proteins have not been associated with cell movement in Drosophila. Mutations in only two of the seven Drosophila Wnt genes, Wingless and DWnt2 (Kozopas et al., 1998), have been reported. Wingless (the ortholog of vertebrate Wntl) signals through the canonical Wnt signaling pathway (Cadigan and Nusse, 1997). The signal transduction mechanism used by DWnt2 has not been characterized.

We have generated mutations in a third Drosophila Wnt gene, DWnt4 (Graba et al., 1995), and show that it facilitates cell movement during ovarian morphogenesis through a unique signaling mechanism that results in FAK accumulation. Our data indicate that a canonical pathway receptor, DFz2 (Bhanot et al., 1999), is the primary receptor for DWnt4 in facilitating cell movement. However, the downstream effector of canonical signaling, TCF (van de Wetering et al., 1997), is not required for DWnt4-mediated cell movement. A mutation in Dsh that specifically disrupts planar polarity (Boutros and Mlodzik, 1999) does disrupt cell movement, but the planar polarity mutants frizzled, prickled, inturned, and fuzzy (Mlodzik, 2000) do not exhibit a DWnt4-like phenotype. Our data therefore indicate that the signaling mechanism used by DWnt4 in this process requires DFz2 and Dsh but is neither the canonical Wnt pathway nor the planar polarity pathway.

### DESCRIPTION OF THE FIGURES

FIG. 1: Wnt treatment increases the protrusive activity of NIH3T3 cells. NIH3T3 cells were plated at low density on fibronectin treated cover slips. After being allowed to re-attach for 12 hours, cells were placed into media that was pre-conditioned either by control 293T cells (A), 293T cells expressing Wnt1 (B) or 293T cells expressing Wnt5a (C and D). After 15 minutes in conditioned media, cells were fixed and stained with Alexa568 conjugated phalloidin to label the actin cyto-skeleton. Wnt treated cells contained higher numbers of actin based protrusions relative to control. These included filopodia (arrows in B and C) as well as membrane ruffles (arrows in D). E. The number of filopodia per cell was counted for NIH3T3 cells treated as described above. Treatment with either Wnt1 or Wnt5a caused a significant increase in the numbers of filapodia per cell relative to control. Columns represent the average number of filopodia per cell. N=50, 80, 76 cells for control, Wnt1 and Wnt5a, respectively. Error bars represent the standard error of the mean for each group shown.

FIG. 2. DWnt4 is expressed in the sheath epithelium throughout its morphogenesis. (A) A cartoon illustrating the development of the Drosophila ovary, modified from King (King, 1970). At 2 hrs APF, the apical cell population (blue) migrates between the terminal filament stacks (red). By 24 hrs APF, the apical cells have divided the germ cells and follicle cells of the central region (green and yellow) into germaria, and half of the basal population (purple) into the basal stalks. At 36 hrs, the apical cell migration is complete and the cells have flattened out to form the squamous epithelium of the ovariolar sheath. By 48 hrs APF, the first egg chambers have pinched off from the germarium (B) A wild type ovary isolated 2 hours APF and stained with antibodies recognizing DWnt4 (red in panels B, C and D) and Engrailed, which labels the terminal filament cells (green in panels B and C). DWnt4 protein is strongly expressed by cells apical to the terminal filaments (apical is up and to the right). (C) A similarly stained ovary isolated 16 hours APF. DWnt4 protein continues to be expressed by the apical cells as they migrate through the central region of the ovary. (D) An ovary isolated 18 hours APF and stained with antibodies recognizing DWnt4 and Fasciclin III, which is expressed by basal stalk cells at this stage (green in panel D). DWnt4 localizes to the apical cells surrounding the newly formed basal stalks. High levels of DWnt4 expression is also observed at this time in the basal-most region of the ovary which will ultimately fuse with cells from the genital disc to form the oviduct. We also observe expression in the adult (not shown) which is restricted to the sheath, the basal half of the terminal filament cells, and regions I and II of the germarium.

FIG. 3. DWnt4 is required for apical cell movement. Wild type (A,E) or DWnt4EMS23 mutant (B-D, F) ovaries were stained with antibodies that recognize laminin (red in all panels except D) or DWnt4 (red in D) and Fasciclin III (green in all panels), which is expressed by basal stalk cells. In wild type ovaries at 18 h APF (A), laminin is concentrated in the basement membrane secreted by the apical cell population (arrows). Lower levels of laminin surround the apical cells, which have divided the basal stalk cells into pre-clusters (asterices). These cells lie in an optical section in the interior of the ovary. In DWnt4 mutants at 18 h APF (B), laminin protein is not observed in a similar optical section through the ovarian interior. Laminin surrounds apical cells in a peripheral section (C), but these cells have not migrated into the ovary and the basal stalk cells have not been divided into pre-clusters (B, C). The failure of these cells to move into the ovary was confirmed using anti-DWnt4 (D). This antibody also reveals apical cells at the periphery but not in the interior of the ovary (compare to FIG. 2D). Nuclei are stained with Hoechst (blue). Wild type apical cells at 24 h APF (E) surround the basal stalks as they form columns. DWnt4 mutant apical cells at this stage (F) are just beginning to move into the interior of the ovary. Apical is up; basal is down.

FIG. 4. DWnt4 is required for FAK to accumulate in focal complexes. Wild type (panels A, A′ and A″) and DWnt4^{EMS23} mutant (panels B, B′ and B″) ovaries were isolated at 2 h APF and stained with anti-FAK (red; A, A″, B, B″) (Fujimoto et al., 1999). Samples were also treated with labeled phalloidin, which binds to filamentous actin (green; A′, A″, B′, B″). In wild type apical cells (A), FAK accumulates in focal complexes (arrow heads) located at the tips of actin-based triangular projections (arrows) (A′, A″). In the apical cells of DWnt4 mutants, the level of FAK is severely reduced (B). FAK fails to accumulate into foci, even where actin-based projections are visible (B′, B″, arrows). Apical is up.

FIG. 5. DWnt4 signals through DFz2 but is TCF independent. (A) DFz2^{C1}/Df(3L)DFz2 adults exhibit phenotypes similar to DWnt4 mutants. Staining with anti-FasIII (green; nuclei are blue) reveals the sheath (arrows) surrounding folded ovarioles (G, germarium). The female sterility associated with the DFz2^{C1} allele has been proposed to be the result of an unrelated mutation on the chromosome (Chen and Struhl, 1999). However, DFz2^{C1} mutants display an identical phenotype when either homozygous or placed over a deficiency. In addition, the DFz2^{C1} phenotype can be partially rescued by ubiquitous expression of a DFz2 transgene. In the presence of the β-tub-DFz2 transgene, the percentage of abnormal ovarioles is reduced to 31% (63/202), indicating that the mutant phenotype is due to loss of DFz2. Rescued females lay fertilized eggs; we presume that the lack of full rescue reflects inappropriate levels of expression from the transgene. In mutants exhibiting partial penetrance of the phenotype, the morphologically normal ovarioles are generally exterior to those that are flopped. (B-E) Clones of cells expressing either DFz2-GPI (B,C) or TCFAN (D,E) were followed by the presence of co-expressed GFP (green in B-E). DFz2-GPI expressing cells (B, C) remain clumped in the apical region and do not move between terminal filament stacks (B), and FAK spots cannot be detected at higher magnification (C). In contrast, TCFAN expressing cells move in between terminal filament stacks (asterices; D) and FAK accumulates within them (E, arrows). In the siblings of the flies from which these ovaries were dissected, we observed frequent wing nicks, indicating that Wg signaling was disrupted.

FIG. 6. DWnt4 signals through Dsh but not other planar polarity components. Adult ovaries from dsh¹/dsh^{vA153} (A) but not fz^{P21}/f^{H51} (B) mutants exhibit phenotypes similar to DWnt4 mutants. Staining with anti-FasIII reveals the sheath surrounding the ovarioles in A (arrows; G, germarium). The germarium and young egg chambers are folded beside an older egg chamber (*). The normal linear arrangement of the ovariole is maintained infz muants (B; see Table 1). The sheath in B is visualized with an antibody to phosphorylated tyrosine, which marks cell membranes. (C-E) Ovaries 2 h APF stained with anti-FAK antibody (pink) and phalloidin (green). Wild type (C, C′) ovaries at 2 hr APF stained with FAK (C) exhibit punctate focal complex staining in the apical cells as they migrate (arrows). Visualization of F-Actin (C′) in addition to FAK shows the position of the terminal filaments (asterices). At this magnification (compare to FIG. 4), the actin-based lamellipodia in the apical cells are not recognizable. dsh¹ (D, D′) and DFz2^{C1} (E, E′) mutant ovaries fail to accumulate FAK. Asterices mark the position of terminal filament stacks stained with phalloidin (D′, E′).

FIG. 7. DWnt4 does not signal through the planar polarity pathway. (A) Fmi protein in wild type ovaries 0-4 hr APF is membrane associated but is not polarized. (B) Dsh-GFP localizes to the cell membrane and throughout the cytoplasm in wild type ovaries 0-4 hr APF. In addition, we observe small spots of Dsh-GFP (arrows), but these do not colocalize with FAK (not shown). (C) Adult ovaries of the indicated genotypes were scored for the percentage of ovarioles that were flopped. A minimum of 100 ovarioles were scored for each genotype. (D) A clone of PKCi expressing cells, marked by coexpression of nuclear GFP, was stained for FAK (pink) and actin (not shown; the position of the terminal filaments is indicated by asterisks). The PKCi-expressing apical cells were most frequently apically situated, but as shown here, can also be found to have moved between the terminal filament stacks. The cells appear to be less motile, however, based on their rounder shape and the fact that FAK spots are reduced. Some FAK spots are present, however (arrow).(E) When PKCi is expressed throughout much of the apical cell population with ptcGAL4, the reduced FAK staining is more apparent. The terminal filaments (asterisks) are marked by the actin at their edges (green). The level of actin staining has been reduced electronically to improve the visibility of the FAK signal. (F) The expression of ptcGAL4 in the ovary 0-4 hr APF, as marked by UAS-GFP; the position of the terminal filaments is indicated with askterisks as marked by actin staining (not shown). In all other tissues examined, ptc is expressed ubiquitously but is upregulated in those cells responding to hh.

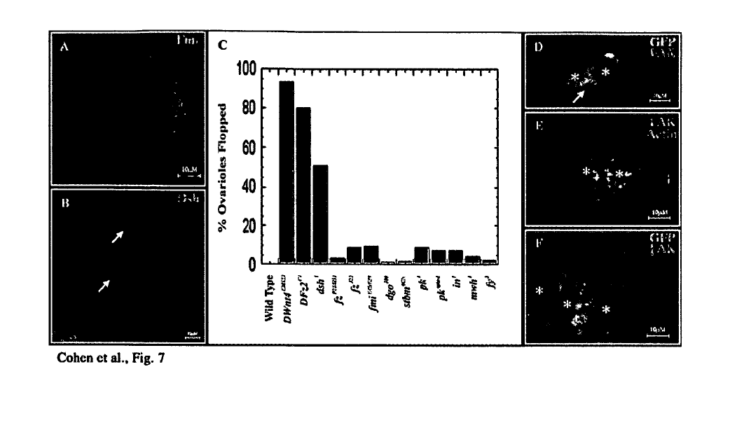

### DETAILED DESCRIPTION

Each of the references (publication, article, web page, information source, GenBank or SwissProt sequence, or patent document, for example) referred to in this specification is hereby specifically incorporated herein by reference, in its entirety. References may be referred to in this document by first author and year, and a complete citation to the references is found at the end of the specification. Furthermore, each reference or any combination of references can be relied on and used, in whole or in part, to make, use, and test embodiments of the invention or specific examples described here. As this statement applies to each and every reference, document, or source of information, this specification will not repeat the incorporation by reference. This statement operates to effectively incorporate by reference in their entirety each and every reference (as defined above) listed or referred to in the specification.

In making and using aspects and embodiments of this invention, one skilled in the art may employ conventional molecular biology, cell biology, virology, microbiology, and recombinant DNA techniques. Exemplary techniques are explained fully in the literature and are well known in the art. For example, one may rely on the following general texts to make and use the invention: Sambrook et al., Molecular Cloning: A Laboratory Manual, Second Edition (1989) Cold Spring Harbor Laboratory Press, Cold Spring Harbor, N.Y. and Sambrook et al. Third Edition (2001); DNA Cloning: A Practical Approach, Volumes I and II (D. N. Glover ed. 1985); Oligonucleotide Synthesis (M. J. Gaited. 1984); Nucleic Acid Hybridization (B. D. Hames & S. J. Higgins eds. (1985)); Transcription And Translation Hames & Higgins, eds. (1984); Animal Cell Culture (RI. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (2001), Coligan et al. (eds.), Current Protocols in Immunology, John Wiley & Sons, Inc. (2001); Dracopdi et al., Current Protocols in Human Genetics, John Wiley & Sons, Inc. (2001), W. Paul et al. (eds.) Fundamental Immunology, Raven Press; E. J. Murray et al. (ed.) Methods in Molecular Biology: Gene Transfer and Expression Protocols, The Humana Press Inc. (1991); and J. E. Celis et al., Cell Biology: A Laboratory Handbook, Academic Press (1994).

DWnt4 is most closely related to vertebrate Wnts 9, 14, and 15 (Bergstein et al., 1997; Graba et al., 1995). Therefore, the results we discuss with DWnt4 can be directly correlated to the mammalian Wnts 9, 14, and 15. We show that the same process of effecting cell motility characteristics, for example, occur in the human cancer cell line NIH3T3. Thus, mammalian wnt pathways behave similarly to the model system in Drosophila. We have used Drosophilia here to take advantage of the genetic work already performed on Wnt systems in Drosophila. The primary feature that distinguishes vertebrate and mammalian Wnt 9, 14, and 15 proteins from other Wnt family members is an unusual spacing of conserved cysteine residues at the C termini of each protein. This feature, and the observation that DWnt4 can elicit a response that is distinct from that of Wingless in the embryonic epidermis, led us to postulate that DWnt4 signals through a noncanonical signaling mechanism (Buratovich et al., 2000; Gieseler et al., 1999; Graba et al., 1995).

To analyze DWnt4 signaling genetically, we generated mutations at the locus (see Experimental Procedures). This resulted in the isolation of 3 mutant alleles, all of which display partial lethality. The cause of lethality has not been determined; embryos hatch with no obvious defects, but the larvae die in the first or second instar. Approximately 10-15% of the mutant individuals survive to adulthood. Sequence analysis of genomic DNA from the mutants reveals that two of the alleles have changes in the DWnt4 coding region: a stop signal is generated at position 343 in DWnt4EMS23 and a 3 bp deletion removes a highly conserved glutamate residue at position 299 in DWnt4C1. The full length protein is 539 amino acids. For each of these alleles, the lethality of DWnt4/DWnt4 is similar to that of DWnt4/Df, indicating that each allele is either amorphic or strongly hypomorphic. The third allele, DWnt4×1 does not contain a mutation in the coding region, suggesting that this allele contains a regulatory mutation. The DWnt4 mutant individuals that survive to adulthood are male and female sterile. Here we have focused on the phenotype in the developing ovary.

Ovariolar Structure is Disrupted in DWnt4 Mutants.

The Drosophila adult ovary contains between 13 and 16 chains of developing egg chambers called ovarioles, each of which is contained within an ovariolar sheath (King, 1970). The sheath that covers each ovariole is composed of a layer of squamous epithelium surrounded by bands of muscle. The sheath epithelium secretes a thick basement membrane which provides structural support to the ovariole.

A specialized group of cells at the apical tip of the germarium, known as the terminal filament cells, are contiguous with the sheath epithelium. Immediately basal to the terminal filaments are germ line and somatic stem cells. The germ line stem cells divide to produce the oocyte and supporting nurse cells; the somatic stem cells give rise to a layer of follicle cells that surround the germ cells (Spradling, 1993). When the oocyte and nurse cells become enveloped by the follicle cells, they pinch off from the germarium to form an egg chamber, and oogenesis proceeds. This process is repeated so that each ovariole contains a chain of egg chambers with the germarium and young egg chambers at the apical end and mature oocytes at the basal end.

The structure of the ovarioles is disrupted in DWnt4 mutants. Wild type ovarioles exhibit a linear arrangement, with their germaria meeting at the apical tip of the ovary and mature oocytes at the basal end. In DWnt4 mutants, the germaria and younger egg chambers “flop” down beside the older cysts, frequently becoming positioned adjacent to the dorsal appendages of mature oocytes. We postulated that the disorganization of the mutant ovarioles results from a lack of structural support from the ovariolar sheath. We examined the sheath epithelium using an antibody against Fasciclin III, which marks the sheath epithelium and the follicle cells in the adult. In wild type, the sheath surrounds the ovariole as a glove surrounds a finger. In DWnt4 mutant females, the sheath is present but the ovariolar “finger” is folded, as if the “glove” is too small. When the sheath is manually removed, however, the ovarioles straighten out and are grossly normal. This suggests that the aberrant structure of the mutant ovaries is due to defects within the ovariolar sheath. To determine how DWnt4 contributes to the structure of the sheath, we first examined its expression during sheath morphogenesis.

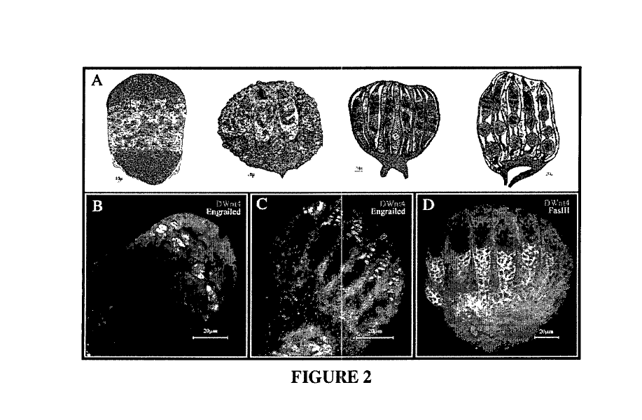

DWnt4 is expressed in cells that migrate to form the ovariolar sheath epithelium. Ovarian morphogenesis begins in the third larval instar and continues through pupal stages (King, 1970) (FIG. 2A). At 2 h after puparium formation (APF), the ovary is organized into four distinct cell populations. Germ cells and follicle cell precursors are located in the central region of the ovary (green and yellow, respectively). The terminal filament cells (red) are organized into stacks (Godt and Laski, 1995; King, 1970). The apical cell population (blue) begins to migrate basally between terminal filament stacks, secreting a thick basement membrane as it moves (King, 1970). Prior to and during their migration, the apical cells maintain close contact with each other and exhibit a roughly cuboidal morphology. By 24 h APF, the migrating apical cells have separated the central region of the ovary into individual germaria and have begun to divide the basal cells (pink) into distinct clusters called basal stalk precursors. By 36 h APF apical cell migration is complete, and the cells flatten to assume their final squamous morphology. By 48 h APF, the first egg chambers have pinched off from the germaria.

DWnt4 is expressed in the migrating apical cells. At 2 h APF, it is expressed in the apical cells (red) as they migrate basally between terminal filament stacks (green; FIG. 2B). In ovaries isolated 16 h APF, DWnt-4 is localized to the migrating cells on either side of the terminal filaments and developing germaria (FIG. 2C). By 18 h APF, DWnt4-expressing cells are observed in between the individual basal stalk clusters (green; FIG. 2D). These data show that DWnt4 is present throughout the apical cell population during ovarian morphogenesis.

DWnt4 is Required for Apical Cell Migration.

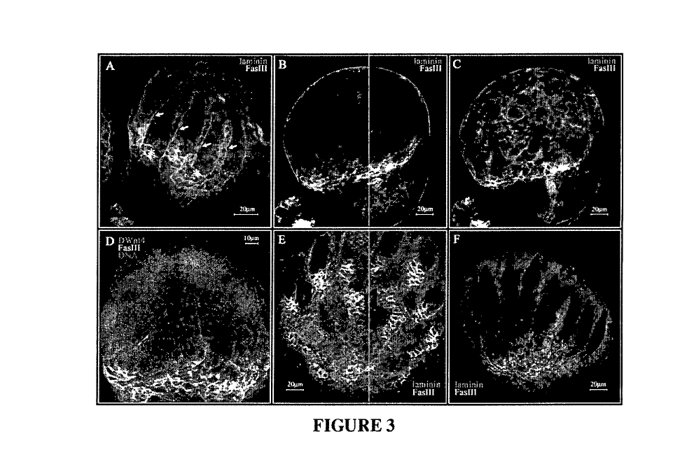

The expression of DWnt4 in the apical cell population led us to examine the behavior of these cells during ovariolar sheath morphogenesis. Two markers for the behavior of the apical cells are laminin, which is present in the basement membrane secreted by these cells as they migrate, and DWnt4 itself. In wild type ovaries isolated 16 h APF, laminin is highly concentrated in the basement membrane (FIG. 3A). Lower levels of laminin are also observed surrounding individual apical cells. At this time, the apical population has migrated between clusters of basal cells.

In DWnt4 mutant ovaries isolated 16 h APF, laminin-secreting cells are absent from the interior of the ovary (FIG. 3B). Laminin secreting cells are observed at the periphery of the ovary, but these cells have failed to move between the basal cells (FIG. 3C). When the apical cells are visualized with the DWnt4 antibody, we also observe cells at the periphery of the ovary but not in the interior (FIG. 3D). This contrasts with the wild type, in which DWnt4 expressing cells separate clearly defined germaria (refer to FIG. 2D). These data suggest that movement of the apical cells is disrupted in DWnt4 mutants. However, since an epithelial sheath ultimately forms in mutant adults, we examined the apical cells 8 h later. At this stage in wild type ovaries, the basal stalks are well separated by the apical cells and are undergoing further morphogenesis to form columns (FIG. 3E). In DWnt4 mutants, sparse rows of laminin-secreting cells have begun to move into the central region of the ovary (FIG. 3F) but have not begun to divide the basal cells. This indicates that limited cell movement does occur in DWnt4 mutant ovaries. However, the paucity of moving cells and the delay in their movement results in epithelial sheaths that are not adequate to fully cover the mature adult ovariole.

The expression of DWnt4 throughout the moving population of cells and the observation that the limited movement that does occur is in the appropriate direction suggests that the role of Wnt signaling is not to provide a polarized cue for movement. An alternative role is suggested by the observation that interruption of Wnt signaling inhibits the stabilization of lamellipodia at the leading edges of moving cells (Wallingford et al., 2000). Rather than providing a polarizing cue to specify direction of movement, Wnt signaling could stabilize the lamellipodia by promoting attachment between the cytoskeleton and the extracellular matrix. Since the role of focal adhesions is to provide such an anchor, we examined the localization of FAK as a marker of focal adhesions in migrating apical cells.

FAK Fails to Accumulate in DWnt4 Mutant Ovaries

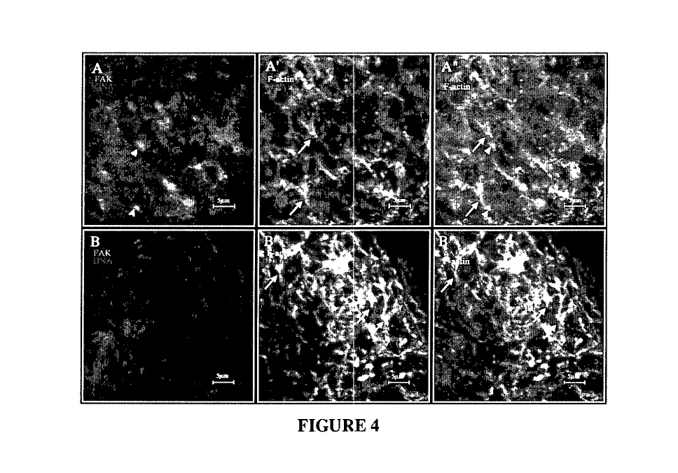

In wild type ovaries at 2 h APF, FAK is present diffusely throughout the cytoplasm but also accumulates in bright spots (FIG. 4A). These spots lie at the tips of V-shaped actin structures (FIG. 4A′, A″) and so appear to represent focal complexes. In DWnt4 mutant ovaries, the level of diffuse FAK within the apical cell population is reduced, and FAK fails to accumulate in spots (FIG. 4B); however, we do see V-shaped actin-enriched structures (FIG. 4B′, B″), suggesting that DWnt4 mutant cells can extend lamellipodia.

The absence of FAK can explain the failure of apical cell migration in DWnt4 mutants, since FAK is required for cell motility in other contexts (Furuta et al., 1995; Ilic et al., 1995; Ilic et al., 1996). Our data indicate that Wnt signaling promotes FAK accumulation to facilitate cell movement. We therefore sought to determine whether this occurs through the canonical Wnt signaling pathway, the planar polarity pathway, or a unique signaling mechanism.

DWnt4 Signals Through DFz2 but is TCF-Independent

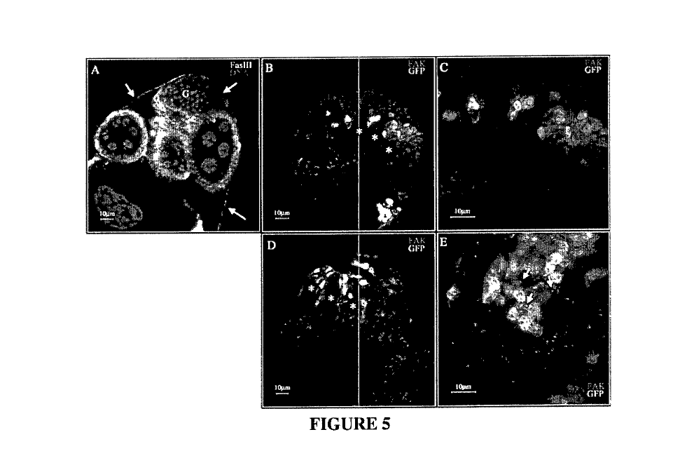

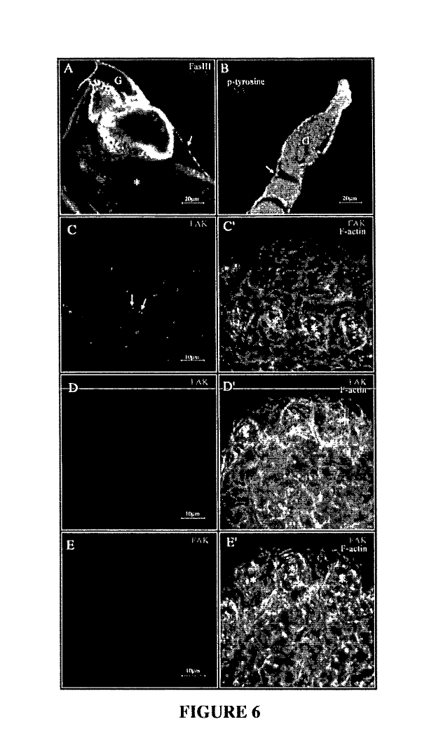

To determine whether DWnt4 signals through the canonical Wnt signaling pathway, we tested whether canonical pathway-specific components are required in migrating apical cells. DFz2 functions redundantly with Fz in canonical signaling but plays no role in planar polarity signaling (Chen and Struhl, 1999; Boutros et al., 2000). DFz2C1 homozygotes are adult viable but sterile (Chen and Struhl, 1999). The adult ovaries exhibit a “flopped” phenotype like those of DWnt4 mutants, suggesting that DFz2 functions as a DWnt4 receptor (FIG. 5A; see Table 1). We also examined clones of cells expressing a dominant negative form of DFz2 [DFz2-GPI] (Zhang and Carthew, 1998). This form of DFz2 interrupts Wg signaling but does not produce planar polarity defects. DFz2-GPI expressing apical cells are found clustered in the apical region of the ovary (FIG. 5B). Furthermore, when FAK is examined in these ovaries, spots of FAK are not observed in cells expressing DFz2-GPI (FIG. 5C). This result confirms that the DFz2C1 adult phenotype is due to a lack of apical cell movement (see also FIG. 6E). It also demonstrates that DFz2 is required in the migrating apical cells to receive the DWnt4 signal. Since DWnt4 is both expressed in and required by the migrating apical cells, it functions as an autocrine signal for cell movement.

D-TCF is a transcription factor that mediates Wg signaling but is not involved in planar polarity signaling (van de Wetering et al., 1997). Individuals lacking D-TCF die as embrys due to loss of Wg signaling. To examine its potential role in cell movement, we therefore generated clones of cells that express an interfering form of this protein [TCFDN] (van de Wetering et al., 1997).In contrast to DFz2-GPI, TCFDN does not interrupt cell movement. Clones of cells expressing TCFDN move between terminal filaments (FIG. 5D), and FAK dots are readily observed within the cells (FIG. 5E). This demonstrates that TCF-mediated transcription is not required for apical cell migration. Therefore, the effect of DWnt4 and DFz2 on apical cell movement is achieved via a noncanonical, TCF-independent mechanism.

The DWnt4 Signaling Pathway is Distinct from the Planar Polarity Pathway.

Although neither DWnt4 mutants nor DFz2 mutants exhibit a planar polarity phenotype, they could engage the planar polarity pathway in the context of moving cells. To determine whether this is the case, we examined mutants that are defective in planar polarity signaling. dsh¹ encodes a protein that transduces canonical signals but is defective in planar polarity signaling (Klingensmith et al., 1994; Thiesen et al., 1994). dsh¹ mutants are fertile, but we noticed that they exhibit reduced fecundity. When we examined the ovaries from these females, we observed a DWnt4-like phenotype (FIG. 6A; see Table 1). The penetrance of the phenotype is reduced compared to DWnt4, presumably due to the fact that Dsh¹ retains some signaling ability in the planar polarity pathway (Boutros et al., 1998). The flopped ovarioles observed in dsh¹ mutants indicate that Dsh is required for DWnt4 signaling in cell movement, and that the dsh¹ mutation disrupts DWnt4 signaling. To confirm that the flopped ovary phenotype of dsh¹ mutants is due to failed cell movement, we examined FAK in dsh1 pupal ovaries (FIG. 6D, D′) and compared them to wild type (FIG. 6C, C′) and DFz2^{C1} (FIG. 6E, E′) mutants. FAK spots are visible in the wild type apical cells, but are absent in both dsh¹ and DFz2^{C1} mutants. Therefore, the dsh¹ lesion disrupts DWnt4 signaling through DFz2. This is consistent with the idea that the planar polarity pathway is engaged by DWnt4 and DFz2, but to test this further, we examined other planar polarity mutants.

Fz is the planar polarity receptor (Adler, 1992). Although loss of Fz activity in planar polarity signaling produces a phenotype similar to dsh1 mutants, we see only minor defects in fz mutant ovaries (FIG. 6B, Table 1). This suggested that the DWnt4 cell movement pathway uses a different receptor from the planar polarity pathway, but that it merges with planar polarity signaling at the level of Dsh. To address this possibility, we examined additional planar polarity mutants. A subset of the planar polarity mutants exhibit planar polarity defects in all tissues examined (referred to as “primary polarity genes”), while others affect polarity only in certain contexts (referred to as “secondary polarity genes”) (Mlodzik, 2000). Fz, Dsh, and Prickled (Pk) are included in the primary group, while Inturned (In) and Fuzzy (Fy) are included in the secondary group. We have examined the adult phenotype of prickled, inturned, and fuzzy mutants (Table 1). These mutants display only minor disruption of ovarian morphology, indicating that DWnt4 does not signal through the planar polarity pathway to facilitate cell movement.

DWnt4 is the first Wnt protein in Drosophila shown to influence cell movement or to signal through a noncanonical mechanism. In the establishment of planar polarity and in cell movement, Wnt signals have been proposed to provide a polarizing cue through a similar noncanonical pathway (McEwen and Peifer, 2000; Tada and Smith, 2000; Wallingford et al., 2000). The function of DWnt4 during cell movement allows us to relate Wnt activity in cell movement to other factors that participate in this process, and to directly compare the cell movement pathway to the planar polarity pathway. Our data reveal first that Wnt signaling faciliates movement by promoting FAK accumulation rather than by polarizing the cells, and second that the pathway employed by DWnt4 is distinct from both the canonical Wnt pathway and the planar polarity pathway.

DWnt4 Facilitates Cell Movement Through FAK

Since Wnt proteins are secreted, they are attractive candidates for directional signals. However, our data are inconsistent with a role for DWnt4 in providing a directional cue. DWnt4 is expressed throughout the population of moving cells and acts as an autocrine signal for these cells; it cannot therefore be the primary polarizing signal. The conclusion that Wnt signaling does not provide direction to cell movement is also supported by the observation that loss of Wntl 1-mediated movement during zebrafish gastrulation can be rescued by ubiquitous expression of Wnt11 (Heisenberg et al., 2000).

Our data indicate that, rather than polarizing the cells, the role of DWnt4 during cell movement is to promote FAK accumulation in focal complexes. In cultured cells, FAK is localized to both focal contacts, which form at the tips of lamellipodia or filopodia, and to focal adhesions, which form at the ends of stress fibers located behind the leading edge of the cell (Parsons et al., 2000). FAK is activated upon localization to focal complexes and focal adhesions. This leads to phosphorylation of multiple targets and ultimately to focal adhesion disassembly, allowing the cell to move forward (Igishi et al., 1999; Parsons et al., 2000; Turner, 2000). FAK is recruited to focal complexes when lamellipodia extend and establish contact with the extracellular matrix through integrin/matrix binding. Our data indicate that Wnt signaling is necessary for this recruitment to occur. In support of our observations, mouse Dsh localizes to focal adhesions in response to Wnt signaling in embryonic kidney cells (Torres and Nelson, 2000). Wnt signaling in these cells regulates the transition from mesenchymal morphology to epithelial morphology. This suggests that Wnt regulation of FAK accumulation may be relevant for cell and tissue organization as well as for cell movement.

DWnt4 Signals Through a Noncanonical Pathway that Includes DFz2

Loss of DFz2 has no impact on planar polarity signaling whereas Fz is required for it (Boutros et al., 2000; Rulifson et al., 2000). Replacing the cysteine rich ligand binding domain of DFz2 with that of Fz does not confer upon DFz2 the ability to alter planar polarity. This has led to the suggestion that the intracellular domain of DFz2 can only engage the canonical pathway. Similarly, the dsh1 mutation has been thought to specifically interfere with signaling from Fz. Our data show that neither of these suggestions are the case; DFz2 can engage a noncanonical, cell movement pathway given the appropriate ligand and cellular environment, and dsh1 interferes with DFz2 signaling in this context.

Although dsh1 interferes with cell movement, the cell movement pathway is distinct from the planar polarity pathway. The planar polarity mutants, fz, pk, in, and fy have marginal effects in the ovary. The low penetrance defects that have been observed in these mutants indicates that they play only a peripheral role at best in cell movement. In contrast, Fz and Pk play fundamental roles in planar polarity, since they function in the eye and leg as well as in the wing and notum. In and Fy are novel proteins that function only in the wing, abdomen, and notum to regulate hair and bristle orientation. Since none of these mutants exhibit significant defects in the ovariolar sheath, the process of determining polarity within an epithelial plane seems to be fundamentally distinct from Wnt facilitation of cell movement.

Protein Kinase C has been implicated in Wnt regulation of vertebrate gastrulation movements (Kuhl et al., 2000), and it is possible that the cell movement pathway diverges from the planar polarity pathway to include regulation of PKC. Given the observation that PKC can regulate FAK activation and/or accumulation in fibroblasts (Mogi et al., 1995; Parsons et al., 2000), specific targeting of Protein Kinase C and its effect on Wnt regulation in mammalian cells can lead to additional methods for directly modifying cell motility characteristics.

The Rho family of small GTPases functions in planar polarity and has a well established role in the regulation of cell movement, so they are obvious candidates for downstream mediators of Wnt signaling. Alternatively, they may function in parallel to DWnt4 to regulate FAK accumulation. An observation that may be important in elucidating the relationship between Cdc42/Rac/Rho and Wnt signaling in motile cells is that actin protrusions appear to form in both DWnt4 mutants (FIG. 4) and in Xenopus cells with disrupted Dsh activity (Wallingford et al., 2000). This suggests that extension of actin filaments and localization of FAK to their ends are two separable events. Cdc42 and Rac regulate actin polymerization and the protrusion of filopodia and lamellipodia at the leading edge of the cell, whereas Rho is activated behind the leading edge and induces formation of actin stress fibers (Chrzanowska-Wodnicka and Burridge, 1992; Critchley, 2000; Parsons et al., 2000). Lack of Rho prevents localization of focal adhesion components to focal adhesions, but it also prevents stress fiber formation (Fincham et al., 1996). The observation that actin-rich protrusions form in the absence of Wnt signaling but FAK fails to accumulate is suggestive of parallel functions for DWnt4 and small GTPases, but further genetic analyses of these factors are necessary to clarify their relationships.

Wnt Proteins Regulate FAK Accumulation and Other Focal Adhesion Proteins

Although we have examined Wnt regulation of FAK accumulation in the context of model systems and specific mammalian systems and cells, the near-ubiquitous expression of both FAK and Wnt family members suggests that regulation of FAK accumulation in focal complexes by Wnt proteins is be widespread. Focal adhesions and FAK are key regulators of cell movement and adhesion to the extracellular matrix in many contexts. They relay signals from the extracellular matrix for cell survival, migration, and proliferation.

Our demonstration that Wnt signaling can regulate FAK and other cytosleletal protein accumulation shows that Wnt proteins exert some of their many effects through regulation of focal complexes and focal adhesions. This has implications not only for development but also in those adult tissues where Wnt signaling may regulate cell morphology, viability, migration, or proliferation. As a primary example, the metastatic process involves cell motility and the changes in a cell's motility characteristics. The methods described here can be adapted with specific cell types to analyze changes in cell motility characteristics that are directkly relevant to metastisis, the control or regulation of metastasis, and prognostic and diagnostic methods related to cancer in humans and mammals. In particular, the Wnt pathway's importance in gastrulation demostrates that the methods here can be combined with ovarian cancer cells, and metastatic events involving ovarian cancers, to develop particular prognostic and diagnostic assays for and methods for determining treatments for ovarian cancer.

### EXAMPLES

Production of Wnt Polypeptides and Fragments Thereof.

We can use Wnt conditioned media as a source of Wnt polypeptides and fragments of them in testing the ability of a cell to modify its cell motility characteristics. Human or mouse, for example, Wnt encoding sequences, such as those encoding mouse Wnt 1, mouse Wnt 5A, human Wnt 9, human Wnt 14, and human Wnt 15, can be inserted into an appropriate mammalian expresison vector. After introducing the vector into a mammalian cell, such as human 293 cells, the growth media collected from the surface of the culture can be used directly as a source of Wnt polypeptides and fragments of Wnt polypeptides. These polypeptides and fragments are known to interact with Wnt receptors on mammalian cells.

To demonstrate that Wnt polypeptides and fragments can modify the cell motility characteristics of a mammalian cell, we treated mammalian cancer cells, NIH3T3, with Wnt conditioned media as discussed above. A number of different time points can be selected to test for cell motility changes. We chose 15 miunutes to demonstrate the fast and specific action of the Wnt polypeptides.

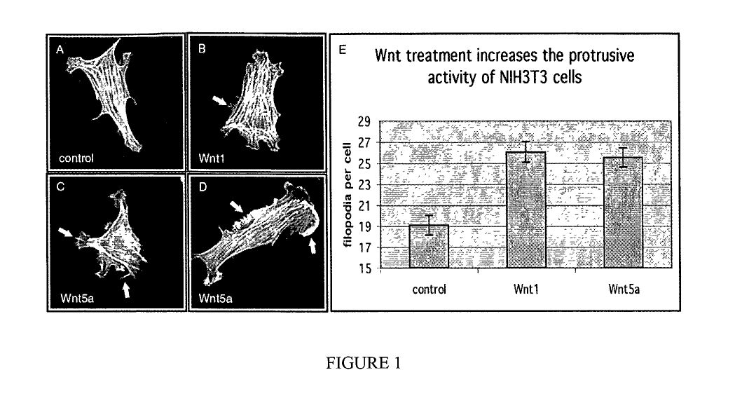

In FIG. 1, we show the human cancer cells after the Wnt treatment. In panels C and D in particular, antibodies to the cytoskeletal protein actin are used to illustrate increased filopodia formation. The increased numbers of filopodia mean these cells are now atively forming the cell structures to move, or the cell motility characteristics have changed from adherant to motile. The degree of motility can be measured through the number of filopodia, for example, or through assyas that measure the levels or increases in cytoskeletal proteins. A number of cytosleletal proteins can be seletected in such an assay, including any one or more of actin, fillimentin, FAK, PAK, src, paxillin, talin, vinculin, tensin, csk, crk, cas, pyk2, and integrins.

Quantitave anitbody assyas can also be performed as known in the art. Cell sorting techniques based on antibody binding can also be performed as known in the art. Antibodies to cytoskeletal proteins can be purchased commercially from a number of sources, including Santa Cruz Biotechnology and Cell Signal Technology.

Various other cell lines can be assyaed as we have shown for the human cancer cell line NIH3T3. One of skill in the art is familiar with numerous cancer cell lines and with methods to harvest and culture primary cancer cells for use in the assys and methods we describe here. In a preferred example, ovarian cancer cells can be cultured or selected for use. Various states of metastatic development or progression can be measured through the new use of Wnt signalling pathways and the compounds and polypeptides and fragments of polypeptides that may interact with the Wnt signalling pathway, and specifically the receptors of the Wnt signalling pathway. Thus, methods where one tests a number of compounds by contacting the compounds with a cell, and then measuring the change in cell motility characteristics of the cell, and determining the changes in the cell structure of protein levels effected by the Wnt pathway can be devised. A primary example of the type of compounds that can be tested are Wnt inhibitors. Inhibitors of the Wnt signalling pathway would inhibit increases in cell motility chracteristics of the cell. Accordingly, Wnt pathway inhibitors can be useful agents in treatments for cancer or reducing metastatic or cell proliferative events. Various human diseases, inlcuding cancer and macular degeneration, involve cell proliferation. Thus, the methods of the invention can be used to test for new drug candidates by using the heretofor unknown correlation between Wnt pathways and focal adhesions in cells.

Use of Model Systems—Drosophila Stocks

Df(2L)RF, Df(2L)ade3, and Df(2L)DE were obtained from Stanley Tiong and David Nash (Tiong and Nash, 1990). Gary Struhl provided y w hsflp; Sp/CyO; DFz2C1 ri FRT2A/TM2 and the rescue stock y w hsflp; Tub>DFz2/CyO; DFz2C1 ri FRT 2A/TM2 (Chen and Struhl, 1999). These stocks were re-balanced over the compound balancer SM6a;TM6B. This allows mutant larvae and pupae to be distinguished from wild type sibs on the basis of the Tubby mutation on TM6B. The DFz2 deficiency Df(3L)DFz2 was provided by Ken Cadigan (Bhanot et al., 1999) and also rebalanced over this balancer. XX/y ras dshl, y w dshv26 FRT101/FM7, y w hsflp22; Act5C>y+>GAL4, UAS-GFPNLS/CyO, pk cn 1, pksplel, cp in 1, kniri-1 pp, y; mwh 1, and cl fy2 nub2 were obtained from the Bloomington Stock Center. The null dsh allele dshVA153 FRT18A/FM7 was obtained from Steve Cohen. UAS-DFz2GPI was provided by Richard Carthew (Zhang and Carthew, 1998); UAS-TCFDN (dominant negative) was provided by Hans Clevers (van de Wetering et al., 1997). The GAL4 line e22cGAL4 drives fairly low levels of ubiquitous expression in the embryo (Brand, 1997). The UAS-DWnt4 line has been described (Gieseler et al., 1999).

Clones of cells expressing UAS constructs were generated by crossing each stock to flies of the genotype y w hsflp22; Act5C>y+>GAL4, UAS-GFPNLS/CyO (Ito et al., 1997); progeny were heat shocked for 1 hr at 32° C. at second-third instar to enable GAL4 to be expressed. Clones were followed through the expression of UAS-GFP.

Isolation of DWnt4 Mutants and Analysis of Germ Line Clones

DWnt4 lies within the same region of the second chromosome that includes wg (Graba et al., 1995). We mapped the breakpoints of two deficiencies in the region, Df(2L)ade3 and Df(2L)RF (Tiong and Nash, 1990), and found that both have breakpoints within DWnt4. Df(2L)ade3 removes DNA distal to the first DWnt4 exon, and Df(2L)RF removes part of the first intron of DWnt4 and DNA proximal to it, including wg. A third deficiency, Df(2L)DE (Tiong and Nash, 1990), completely removes the DWnt4 locus.

Three independent screens were conducted to isolate mutations that fail to complement these deficiencies. Three semi-lethal alleles of one complementation group were isolated. During a screen for EMS-induced lethal mutations that are uncovered by Df(2L)RF, we obtained one allele, EMS23, that was semi-lethal in combination with all three deficiencies. DWnt4 was the only gene predicted to be affected by all three deficiencies. We isolated additional alleles through two independent screens. In one screen, cn bw sp males were mutagenized with X-rays and crossed to cn bw sp/CyO females. Males of the genotype cn bw sp */CyO (where * indicates a mutagenized chromosome) were crossed to EMS23 cn bw sp/SM5 females. Approximately 8000 flies were screened for failure to complement the original EMS23 mutation. One allele was recovered (C1). In a second screen, w1118 males were mutagenized with EMS and crossed to CyOen 1/sli females. CyOen 11 is a balancer that includes a LacZ insertion at the wingless locus and removes wg activity. Males of the genotype w1118; */CyOen11 were crossed to females of the genotype Df(2L)DE/CyO. Approximately 3000 chromosomes were screened for mutations resulting in failure to complement Df(2L)DE. In this screen, we isolated three alleles that failed to complement the deficiency. Two of these fell within the complementation group distal to DWnt4; one allele (X1) failed to complement all three deficiencies in the region as well as the original EMS23 mutation. This allele exhibits a slightly higher frequency of surviving mutant adults (20-25%), and females occasionally lay eggs. This allele therefore appears to be hypomorphic. For all of the analyses of pupal ovaries, the DWnt4 mutant alleles were re-balanced over SM6a;TM6B.

To confirm that the complementation group defined by the EMS23 mutation corresponded to DWnt4, we sequenced the coding region from EMS23, C1, and X1. Flies carrying each allele were crossed to Df(2L)DE and DNA was prepared from hemizygous escaper adults. Primers were designed at approximately 500 bp intervals along the coding sequence with 50 bp of overlap between primer pairs. The sequences were compared to DNA from the orginal strains, cn bw sp and w1118. Although a few polymorphisms were observed between these strains, none resulted in coding changes. The changes noted in the mutants are described in the text. We were also able to rescue partially the lethality associated with DWnt4 mutations by ubiquitously expressing low levels of DWnt4 through GAL4 mediated expression. For these experiments, DWnt4EMS23 e22CGAL4/CyO flies were crossed to Df(2L)DE/CyO;UASDWnt4. In this experiment, the number of surviving mutants was increased to 60% (92/471 or 20% of the total progeny were hemizygotes, where full rescue would be 33% of the total progeny). The rescue by this GAL4 driver did not include restoration of fertility.

We discovered a sequencing error in the original DWnt4 cDNA sequence. This error resulted in the predicted DWnt4 protein being 137 amino acids shorter than it actually is. We have confirmed this by examining the protein size produced by the full length cDNA transfected into S2 cells to the protein produced by a cDNA with the 5′ end truncated to the original predicted start of translation (not shown). The corrected sequence will be submitted to GenBank.

Germ line clones were generated with the FLP-DFS technique (Chou and Perrimon, 1996) by crossing females of the genotype DWnt4EMS23 or C1 FRT40A/CyO to males of the genotype y w hs-flp22; P{ovoD1} FRT40A/CyO. Progeny were heat shocked at 37° C. for 2 hours as third instar larvae or early pupae. Straight winged females were mated to DWnt4EMS23/CyO males. The females bearing clones were fertile, and their progeny hatched, indicating that DWnt4 is not required in the germline.

Production of DWnt4 Antibody

To generate an antibody against DWnt4, the entire coding region from the originally reported start site was cloned into pET28a (Novagen) to produce a His-tagged protein. This protein was expressed in bacteria and purified on a nickel column. The protein was solubilized in guanidinium hydrochloride and injected into rabbits (Pocono Rabbit Farm). IgG from immune serum was isolated using a Protein A column; the antibody was then preadsorbed with S2 cell acetone powder prior to use. Specificity of the antibody was evaluated by examining S2 cell-expressed proteins on Westerns and by examining expression in DWnt4 deficiency embryos (not shown). In the S2 cell experiments, cells were transfected with a construct bearing the full length DWnt4 cDNA, a construct bearing the cDNA truncated at the originally predicted start of translation, or a construct bearing the wingless cDNA. The antibody detected two bands (presumably multiple glycoforms) centered around 59 Kd in extracts from full length DWnt4 cDNA expressing cells, a pair of bands of approximately 43 Kd in extracts from truncated DWnt4 cDNA expressing cells, and no bands in extracts from Wg-expressing cells. When the pattern of protein was examined in wild type embryos, a pattern similar to that of DWnt4 RNA was detected; in the central nervous system, the protein is localized to axons. In Df(2L)ade3/Df(2L)DE embryos, no protein is detected.

Dissection and Immunofluorescence

Larvae of the desired genotypes were sexed, and females aged in food vials. White pre-pupae were collected every 2 hours. To isolate ovaries 2 hours APF, the posterior half of these flies were immediately dissected, inverted and fixed for one hour with 4% formaldehyde in phosphate buffered saline [PBS]. Once fixed, samples were washed three times with 0.1% Tween-20 dissolved in PBS [PBST] and the ovaries removed using tungsten needles. Ovaries were then placed in collection nets. These nets were made from 2 ml cryo-tubes (externally threaded) that had their bases cut off and holes punched through their caps with an 18 gauge syringe. The caps held 25 mm polycarbonate filters with 8.0 micron pores (Osmonics Cat# K80CP02500). This allowed the ovaries to be retained while liquid was forced out using pressure generated by a syringe barrel. After sufficient numbers of ovaries were isolated, immunofluorescence labeling was performed as described.

For ovaries isolated between 16 and 72 hours APF, white pre-pupae were collected, placed on media filled plates and aged at 25 Co. After incubation, the contents of the pupal cases were emptied into Drosophila Ringers solution and the ovaries isolated with tungsten needles under a dissecting microscope. Ovaries were then placed in 4 well tissue culture dishes, fixed and stained.

Immunofluorescence was performed as described (White, 1998) with the following modifications. All dissections were done in Drosophila Ringer's solution. Samples were fixed with 4% formaldehyde dissolved in PBS. After fixation washes were done with PBST. Samples were blocked with 2% bovine serum albumin [BSA] and 5% normal goat serum dissolved in PBST. All antibodies and anti-sera were diluted in PBST containing 2% BSA and 5% NGS.

The mouse monoclonal antibodies 4D9 anti-Engrailed/Invected and 7G10 anti-Fasciclin III developed by Dr. Corey Goodman were obtained from the Developmental Studies Hybridoma Bank and diluted 1:1 and 1:5, respectively. Rabbit polyclonal anti-sera raised against Drosophila laminin was obtained from Dr Lisa Fessler (Fessler et al., 1987) and diluted 1:500. Guinea Pig polyclonal antisera 1562, raised against Drosophila Dfak56 (Fujimoto et al., 1999), was provided by Dr. Richard Hynes and diluted 1:5000. Rabbit anti-Armadillo was provided by Dr. Eric Wieschaus (Riggleman et al., 1990). Rabbit polyclonal anti-sera against DWnt4 were generated in our laboratory as described above. IgG purified anti-serum was diluted 1:50 and preabsorbed several times with S2 cell acetone powder prior to use. Since S2 cells do not produce DWnt4 protein (data not shown), this step significantly reduces background staining without affecting specific signal.

Alexa488, Alexa556 and Cy5 conjugated secondary antibodies against mouse, rabbit and guinea pig were obtained from Molecular Probes and diluted 1:500 prior to use. When desired, 30 units/ml Alexa556 conjugated phalloidin was added to the diluted secondary antibodies to stain filamentous actin. Prior to mounting, samples were incubated for 10 minutes with 5 mg/ml Hoechst 33342 dissolved in PBST to label cell nuclei.

The invention described and exemplified above is not limited to the specific embodiments and examples presented here. One skilled in the art can use the techniques and knowledge available through the documents and references noted and specifically incorporated herein, or other documents or references, to make and use additional embodiments. Thus, the description above should not be taken as a limitation of the scope or content of this invention.

### REFERENCES INCORPORATED BY REFERENCE

Adler, P. N. (1992). The genetic control of tissue polarity in Drosophila. Bioessays 14, 735-41.

Bergstein, I., Eisenberg, L. M., Bhalerao, J., Jenkins, N. A., Copeland, N. G., Osborne, M. P., Bowcock, A. M., and Brown, A. M. (1997). Isolation of two novel WNT genes, WNT14 and WNT15, one of which (WNT15) is closely linked to WNT3 on human chromosome 17q21. Genomics 46, 450-8.

Bhanot, P., Fish, M., Jemison, J. A., Nusse, R., Nathans, J., and Cadigan, K. M. (1999). Frizzled and frizzled-2 function as redundant receptors for Wingless during Drosophila embryonic development. Development 126,4175-86.

Boutros, M., Mihaly, J., Bouwmeester, T., and Mlodzik, M. (2000). Signaling specificity by Frizzled receptors in Drosophila. Science 288, 1825-8.

Boutros, M., and Miodzik, M. (1999). Dishevelled: at the crossroads of divergent intracellular signaling pathways. Mechanisms of Development 83, 27-37.

Boutros, M., Paricio, N., Strutt, D. I., and Mlodzik, M. (1998). Dishevelled activates JNK and discriminates between JNK pathways in planar polarity and wingless signaling. Cell 94, 109-18.

Brand, A. (1997). FlyBase Personal Communication.

Buratovich, M., Anderson, S., Gieseler, K., Pradel, J., and Wilder, E. L. (2000). DWnt-4 and Wingless have distinct activities in the Drosophila dorsal epidermis. Dev. Genes Evol. 210, 111-119.

Cadigan, K. M., and Nusse, R. (1997). Wnt signaling: a common theme in animal development. Genes & Development 11, 3286-305.

Chen, C. M., and Struhl, G. (1999). Wingless transduction by the Frizzled and Frizzled2 proteins of Drosophila. Development 126, 5441-52.

Chou, T. B., and Perrimon, N. (1996). The autosomal FLP-DFS technique for generating germline mosaics in Drosophila melanogaster. Genetics 144, 1673-9.

Chrzanowska-Wodnicka, M., and Burridge, K. (1992). Rho, rac and the actin cytoskeleton. Bioessays 14, 777-8.

Critchley, D. R. (2000). Focal adhesions—the cytoskeletal connection. Current Opinion in Cell Biology 12, 133-9.

Fessler, L. I., Campbell, A. G., Duncan, K. G., and Fessler, J. H. (1987). Drosophila laminin: characterization and localization. Journal of Cell Biology 105, 2383-91.

Fincham, V. J., Unlu, M., Brunton, V. G., Pitts, J. D., Wyke, J. A., and Frame, M. C. (1996). Translocation of Src kinase to the cell periphery is mediated by the actin cytoskeleton under the control of the Rho family of small G proteins. Journal of Cell Biology 135, 1551-64.

Fujimoto, J., Sawamoto, K., Okabe, M., Takagi, Y., Tezuka, T., Yoshikawa, S., Ryo, H., Okano, H., and Yamamoto, T. (1999). Cloning and characterization of Dfak56, a homolog of focal adhesion kinase, in Drosophila melanogaster. Journal of Biological Chemistry 274, 29196-201.

Furuta, Y., Ilic, D., Kanazawa, S., Takeda, N., Yamamoto, T., and Aizawa, S. (1995). Mesodermal defect in late phase of gastrulation by a targeted mutation of focal adhesion kinase, FAK. Oncogene 11, 1989-95.

Gieseler, K., Graba, Y., Mariol, M. C., Wilder, E. L., Martinez-Arias, A., Lemaire, P., and Pradel, J. (1999). Antagonist activity of DWnt-4 and wingless in the Drosophila embryonic ventral ectoderm and in heterologous Xenopus assays. Mechanisms of Development 85, 123-31.

Godt, D., and Laski, F. A. (1995). Mechanisms of cell rearrangement and cell recruitment in Drosophila ovary morphogenesis and the requirement of bric &#x00E0; brac. Development 121, 173-87.

Graba, Y., Gieseler, K., Aragnol, D., Laurenti, P., Mariol, M. C., Berenger, H., Sagnier, T., and Pradel, J. (1995). DWnt-4, a novel Drosophila Wnt gene acts downstream of homeotic complex genes in the visceral mesoderm. Development 121, 209-18.

Gumbiner, B. M. (1996). Cell Adhesion: The Molecular Basis of Tissue Architecture and Morphogenesis. Cell 84.

Heisenberg, C. P., Tada, M., Rauch, G. J., Saude, L., Concha, M. L., Geisler, R., Stemple, D. L., Smith, J. C., and Wilson, S. W. (2000). Silberblick/Wnt11 mediates convergent extension movements during zebrafish gastrulation. Nature 405, 76-81.

Igishi, T., Fukuhara, S., Patel, V., Katz, B. Z., Yamada, K. M., and Gutkind, J. S. (1999). Divergent signaling pathways link focal adhesion kinase to mitogen-activated protein kinase cascades. Evidence for a role of paxillin in c-Jun NH(2)-terminal kinase activation. Journal of Biological Chemistry 274, 30738-46.

Ilic, D., Furuta, Y., Kanazawa, S., Takeda, N., Sobue, K., Nakatsuji, N., Nomura, S., Fujimoto, J., Okada, M., and Yamamoto, T. (1995). Reduced cell motility and enhanced focal adhesion contact formation in cells from FAK-deficient mice. Nature 377, 539-44.

Ilic, D., Kanazawa, S., Furuta, Y., Yamamoto, T., and Aizawa, S. (1996). Impairment of mobility in endodermal cells by FAK deficiency. Experimental Cell Research 222, 298-303.

Ito, K., Awano, W., Suzuki, K., Hiromi, Y., and Yamamoto, D. (1997). The Drosophila mushroom body is a quadruple structure of clonal units each of which contains a virtually identical set of neurones and glial cells. Development 124, 761-71.

King, R. C. (1970). Ovarian Development in Drosophila melanogaster (New York and London: Academic Press).

Klingensmith, J., Nusse, R., and Perrimon, N. (1994). The Drosophila segment polarity gene dishevelled encodes a novel protein required for response to the wingless signal. Genes & Development 8, 118-30.

Kozopas, K. M., Samos, C. H., and Nusse, R. (1998). DWnt-2, a Drosophila Wnt gene required for the development of the male reproductive tract, specifies a sexually dimorphic cell fate. Genes & Development 12, 1155-65.

Kuhl, M., Sheldahl, L. C., Park, M., Miller, J. R., and Moon, R. T. (2000). The Wnt/Ca2+ pathway: a new vertebrate Wnt signaling pathway takes shape. Trends in Genetics 16, 279-83.

Lauffenburger, D. A., and Horwitz, A. F. (1996). Cell Migration: A Physically Integrated Molecular Pfocess. Cell 84, 359-369.

Liu, S., Calderwood, D. A., and Ginsberg, M. H. (2000). Integrin cytoplasmic domain-binding proteins. Journal of Cell Science 113, 3563-71.

McEwen, D. G., and Peifer, M. (2000). Wnt signaling: Moving in a new direction. Current Biology 10, R562-4.

Mlodzik, M. (2000). Spiny legs and prickled bodies: new insights and complexities in planar polarity establishment. Bioessays 22, 311-5.

Mogi, A., Hatai, M., Soga, H., Takenoshita, S., Nagamachi, Y., Fujimoto, J., Yamamoto, T., Yokota, J., and Yaoi, Y. (1995). Possible role of protein kinase C in the regulation of intracellular stability of focal adhesion kinase in mouse 3T3 cells. FEBS Letters 373, 135-40.

Parsons, J. T., Martin, K. H., Slack, J. K., Taylor, J. M., and Weed, S. A. (2000). Focal adhesion kinase: a regulator of focal adhesion dynamics and cell movement. Oncogene 19, 5606-13.

Patapoutian, A., and Reichardt, L. F. (2000). Roles of Wnt proteins in neural development and maintenance. Current Opinion in Neurobiology 10, 392-9.

Pinson, K. I., Brennan, J., Monkley, s., Avery, B. J., and Skarnes, W. C. (2000). An LDL-receptor-related protein mediates Wnt signalling in mice. Nature 407, 535-538.

Polakis, P. (2000). Wnt signaling and cancer. Genes & Development 14, 1837-51.

Riggleman, B., Schedl, P., and Wieschaus, E. (1990). Spatial expression of the Drosophila segment polarity gene armadillo is posttranscriptionally regulated by wingless. Cell 63, 549-60.

Rulifson, E. J., Wu, C. H., and Nusse, R. (2000). Pathway specificity by the bifunctional receptor frizzled is determined by affinity for wingless. Molecular Cell 6, 117-26.

Spradling, A. (1993). Developmental genetics of oogenesis. In The development of Drosophila melanogaster, M. Bate and A. Martinez-Arias, eds. (Cold Spring Harbor, N.Y.: Cold Spring Harbor Laboratory Press), pp. 1-70.

Tada, M., and Smith, J. C. (2000). Xwntl 1 is a target of Xenopus Brachyury: regulation of gastrulation movements via Dishevelled, but not through the canonical Wnt pathway. Development 127, 2227-38.

Tamai, K., Semenov, M., Kato, Y., Spokony, R., Liu, C., Katsumaya, Y., Hess, F., Saint-Jeannet, J. P., and He, X. (2000) LDL-receptor-related proteins in Wnt signal transduction. Nature 407, 530-535.

Thiesen, H., Purcell, J., Bennett, M., Kansagara, D., Syed, A., and Marsh, J. L. (1994). dishevelled is required during wingless signalling to establish both cell polarity and cell identity. Development 120, 347-360.

Tiong, S. Y. K., and Nash, D. (1990). Genetic-analysis of the adenosine-3 (gart) region of the 2nd chromosome of Drosophila-melanogaster. Genetics 124, 889-897.

Torres, M. A., and Nelson, W. J. (2000). Colocalization and redistribution of dishevelled and actin during Wnt-induced mesenchymal morphogenesis. Journal of Cell Biology 149, 1433-42.

Turner, C. E. (2000). Paxillin interactions. Journal of Cell Science 113, 4139-40.

van de Wetering, M., Cavallo, R., Dooijes, D., van Beest, M., van Es, J., Loureiro, J., Ypma, A., Hursh, D., Jones, T., Bejsovec, A., Peifer, M., Mortin, M., and Clevers, H. (1997). Armadillo coactivates transcription driven by the product of the Drosophila segment polarity gene dTCF. Cell 88, 789-99.

Wallingford, J. B., Rowning, B. A., Vogeli, K. M., Rothbacher, U., Fraser, S. E., and Harland, R. M. (2000). Dishevelled controls cell polarity during Xenopus gastrulation. Nature 405, 81-5.

Wehrli, M., Dougan, S. T., Caldwell, K., O'Keefe, L., Schwartz, S., Vaizel-Ohayon, D., Schejter, E., Tomlinson, A., and DiNardo, S. (2000). arrow encodes an LDL-receptor-related protein essential for Wingless signalling. Nature 407, 527-530.

White, R. A. H. (1998). Immunolabeling of Drosophila. In Drosophila: A Practical Approach, D. B. Roberts, ed. (Oxford: Oxford University Press).

Zhang, J., and Carthew, R. W. (1998). Interactions between Wingless and DFz2 during Drosophila wing development. Development 125, 3075-85.

Katoh, M. (2002). Regulation of WNT3 and WNT3A mRNAs in human cancer cell lines NT2, MCF-7, and MKN45. Int. J. Oncol. 20 (2), 373-377.

We claim:

1. A method for modifying the cell motility characteristic of a cell, comprising causing an activation of or inhibiting an activation of the Wnt pathway of the cell or comprising contacting the cell with a Wnt poplypeptide or fragment thereof, and measuring the morphological changes in or the protein levels of the focal adhesions of the cell, whereby changes in the morphology of the cell including increased or decreased numbers of philipodia indicate a modified cell motility characteristic, and whereby changes in the levels of focal adhesion proteins that are capable of binding to an extracellular matrix indicate a modified cell motility characteristic.

2. A method of claim 1, wherein a Wnt conditioned media comprising one or more Wnt polypeptides or fragments thereof are contacted with the cell.

3. A method of claim 1, wherein the levels of actin in the cell are measured.

4. A method of claim 1, wherein the changes in cell focal adhesion protein levels of one or more of actin, FAK, PAK, src, paxillin, talin, vinculin, tensin, csk, crk, cas, and pyk2 are measured.

5. A method of claim 2, wherein a DNA comprising SEQ ID NO: 2 [wnt 14] or SEQ ID NO: 3 [wn1] is used to prepare Wnt comditioned media.

7. The method of claim 1, wherein one or more compounds are further contacted with the cell.

8. The method of claim 3, wherein one or more compounds are further contacted with the cell.

9. The method of claim 4, wherein one or more further compounds are contacted with the cell.

10. A method of screening one or more compounds for the ability to increase or decrease the cell motility characteristics of a cell, comprising contacting the cell with the one or more compounds, whereby the Wnt signalling pathway is induced or inhibited, and measuring the number of focal adhesions in the cell compared to a control.

11. The method of claim 10, wherein the one or more compound directly binds to a Wnt receptor.

12. The method of claim 11, wherein the cell is a human cancer cell.

13. The method of claim 12, wherein the compound is a polypeptide.

14. The method of claim 10, wherein the measuring the focal adhesions employs an antibody.

15. The method of claim 14, wherein the antibody specifically binds to one or more of more of actin, FAK, PAK, src, paxillin, talin, vinculin, tensin, csk, crk, cas, and pyk2.
